(19) United States
(12) Patent Application Publication
(10) Pub. No.: US 2006/0235006 A1
(43) Pub. Date: Oct. 19, 2006

(54) Combinations, methods and compositions for treating cancer

(76) Inventors: Francis Y. Lee, Yardley, PA (US); Roberto Weinmann, Princeton, NJ (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(21) Appl. No.: 11/402,502

(22) Filed: Apr. 12, 2006

Related U.S. Application Data
(60) Provisional application No. 60/748,433, filed on Dec. 8, 2005.
(60) Provisional application No. 60/670,744, filed on Apr. 13, 2005.

(51) Int. Cl. A61K 31/5513

(52) U.S. Cl. 514/221

(57) ABSTRACT

The invention relates to a combination of BCR-ABL inhibitor, exemplified by ′N-(2-Chloro-6-methylphenyl)-2-[[6-[4-(2-hydroxyethyl)-1-piperazinyl]-2-methyl-4-pyrimidinyl]amino]-5-thiazolecarboxamide and/or other BCR/ABL inhibitors, and a stem cell selective cytotoxic, exemplified by (R)-2,3,4,5-tetrahydro-1-(1H-imidazol-4-ylmethyl)-3-(phenylmethyl)-4-(2-thienylsulfonyl)-1H-1,4-benzodiazepine-7-carbonitrile, hydrochloride salt, and or other stem cell cytotoxic agents, pharmaceutical compositions of the combination and to methods of using the pharmaceutical compositions in the treatment of oncological disorders.

### RELATED APPLICATIONS

This application claims priority benefit under Title 35 § 119(e) of U.S. provisional Application Nos. 60/748,433, filed Dec. 8, 2005, and 60/670,744, filed Apr. 13, 2005, the contents of which are herein incorporated by reference.

The invention relates to combinations for treating cancer, pharmaceutical compositions, and to methods of using the pharmaceutical compositions in the treatment of oncological and immunological disorders.

The compound of formula (I) ′N-(2-Chloro-6-methylphenyl)-2-[[6-[4-(2-hydroxyethyl)-1-piperazinyl]-2-methyl-4-pyrimidinyl]amino]-5-thiazolecarboxamide, is a protein tyrosine kinsase inhibitor, for example a Src Kinase inhibitor, and is useful in the treatment of immunologic and oncological diseases. The compound of formula (I) is also known as dasatinib or BMS-354825. The compound of formula (I) is also an inhibitor of BCR/ABL, and/or ABL inhibitor. Compounds which inhibit Src and/or BCR/ABL are useful in the treatment of cancers such as CML and ALL.

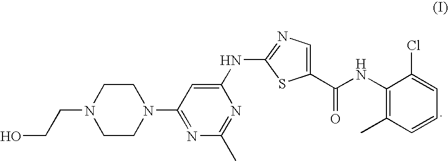

The compound of formula (I) and its preparation have been previously described in U.S. Pat. No. 6,596,746, issued Jul. 22, 2003, which is hereby incorporated by reference. The compound is ideally a crystalline monohydrate form such as described in U.S. patent application Ser. No. 11/051,208, filed Feb. 4, 2005, which is hereby incorporated by reference. Alternatively, the compound of formula (I) may exist in other crystalline forms, either as a neat compound or as a solvate.

The compound of formula (II), (R)-2,3,4,5-tetrahydro-1-(1H-imidazol-4-ylmethyl)-3-(phenylmethyl)-4-(2-thienylsulfonyl)-1H-1,4-benzodiazepine-7-carbonitrile, hydrochloride salt, is an anti-cancer agent. The compound of formula (II) is also known as BMS-214662. The compound of formula (II) is a cytotoxic which is known to kill non-proliferating cancer cells preferentially. The compound of formula (II) may further be useful in killing stem cells.

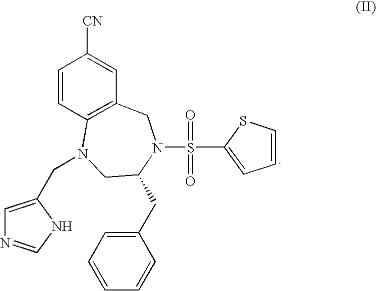

The compound of formula (II), its preparation, and uses thereof are described in U.S. Pat. No. 6,012,029, which is herein incorporated by reference. The uses of the compound of formula (II) are also described in WO2004/015130, published Feb. 19, 2004, which is herein incorporated by reference.

### SUMMARY OF THE INVENTION

Accordingly, an embodiment of the present invention is directed to a combination of the compound of formula (II), a quiescent cell selective cytotoxic, in combination with an BCR/ABL inhibitor.

Additionally, an embodiment of the present invention is directed to a combination including a stem cell selective cytotoxic agents, in combination with a BCR/ABL inhibitor.

Additionally, an embodiment of the present invention is directed to a use of the combination including a stem cell selective cytotoxic agents, in combination with a BCR/ABL inhibitor, for the preparation of a medicament for treating cancer.

An embodiment of the present invention is directed to pharmaceutical compositions comprising a combination of the compound of a pharmaceutically acceptable carrier and a therapeutically effective amount of the compound of a combination of the formula (II) or formula (III) and a BCR/ABL inhibitor.

The invention may be embodied in other specific forms without departing from the spirit or essential attributes thereof. This invention also encompasses all combinations of alternative aspects of the invention noted herein. It is understood that any and all embodiments of the present invention may be taken in conjunction with any other embodiment to describe additional embodiments of the present invention. Furthermore, any elements of an embodiment are meant to be combined with any and all other elements from any of the embodiments to describe additional embodiments.

### BRIEF DESCRIPTION OF THE DRAWINGS

FIG. 1 shows malignant cell growth kinetics and drug sensitivity—hypothesis for synergistic therapeutic potential of dasatinib and BMS-214662.

FIG. 2 shows BMS-214662 affords massive killing of clonogenic tumor cells in vivo, and is specific for non-proliferating cells. (A) Analysis of tumors xenografts by FACS analysis demonstrated that only 20% of tumor cells were proliferative. The vast majority of the tumor cells were in the non-proliferative (G0) growth stage. Non-proliferative cells were identified by prolonged BrdU labeling (24 h) of tumor cells within a solid tumor by continuous infusion of mice bearing the HCT-116 human colon carcinoma subcutaneously in vivo. (B) BMS-214662 killed >90% of clonogenic cells, the vast majority of which would be non-proliferating. (C) BMS-214662 has greater cell killing potency in quiescent than in proliferating cells.

FIG. 3 shows dasatinib is more cytotoxic in proliferating cells (P) compared with quiescent cells (Q). The IC50 of dasatinib in quiescent K562 cells was >11.2 nM compared with 0.69 nM in proliferating K562 cells.

FIG. 4 shows BMS-214662 is more cytotoxic in quiescent cells (Q) compared with proliferating cells (P). BMS-214662 was 68-fold and 4-fold more potent in killing quiescent K562 cells (IC50=0.7 μM) than proliferating K562 cells (IC50=47.5 μM) by a cell growth (A) and clonogenic cell survival assay (B), respectively.

FIG. 5 shows the combination of dasatinib and BMS-214662 has synergistic cytotoxicity against K562 CML cell culture comprising both proliferating and non-proliferating cells. (A) A conservative isobologram shows a high level of synergy between dasatinib and BMS-214662. The position of the central data point relative to the isobologram indicates level of synergy. The further to the left this data point, the greater the synergy. (B) This synergy was corroborated by analysis of combination index (CI). Anything below the CI threshold of 1 is synergistic; anything above this threshold is not. The CI was calculated using CalcuSyn™ software (Cambridge, England).

FIG. 6 shows comparative drug exposure of BMS-214662 in mouse versus human. A dose between 40 and 80 mg/kg BMS-214662 in mice was most comparable to human pharmacokinetics. The figure shows plasma pharmacokinetics following intravenous (IV) bolus injection. Representative human pharmacokinetics are from study CA158003, a 1-hr infusional dose-escalation study of BMS-214662.

FIG. 7 shows dasatinib activity is enhanced by BMS-214662 in vivo. The combination of dasatinib and BMS-214662 produced a superior anti-leukemic activity than either dasatinib alone (P=0.0157) or BMS-214662 alone (P=0.0002) in a mouse CML model. Human tumor xenografts (propagated from CML cell lines) were maintained in Balb/c nu/nu nude or SCID mice and propagated as subcutaneous (SC) transplants. Animals were weighed before treatment initiation (Wt1) and following last treatment dose (Wt2). The difference in body weight (Wt2−Wt1) provides a measure of treatment-related toxicity. Tumor weights (mg) were estimated as follows: tumor weight=(length×width2)/2. Between-group comparison of in vivo efficacy was performed using Gehan's generalized Wilcoxon test.

FIG. 8 shows the BMS-214662 drug exposure needed for enhancing the in vivo efficacy of dasatinib is achievable in humans. This is the case with both (A) 24-hour infusion and (B) 1-hour infusion (CA158-003)

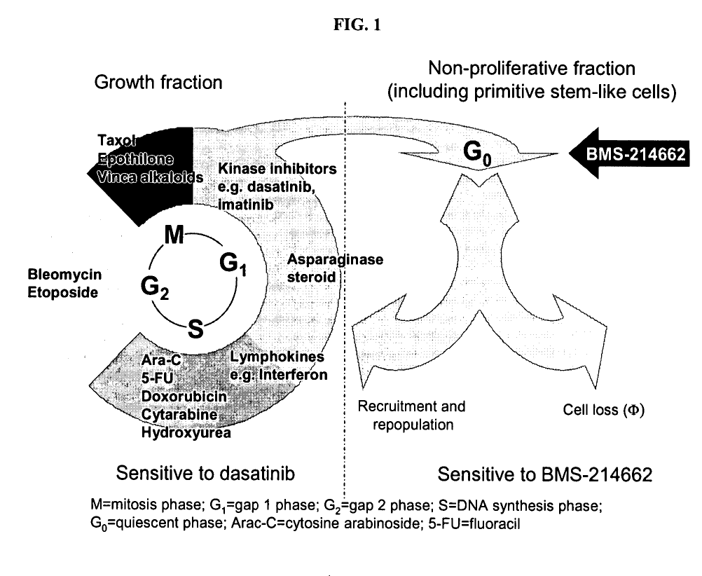

### DETAILED DESCRIPTION OF EMBODIMENTS OF THE INVENTION

In one embodiment of the invention, the invention is directed to a combination of the compound of formula (II), 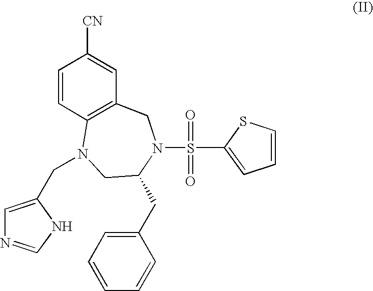
or pharmaceutically acceptable salts thereof,
and an BCR/ABL inhibitor or pharmaceutically acceptable salt thereof.

Alternatively, the invention is directed to a combination of the compound of formula (m) 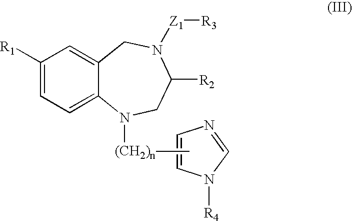
or a pharmaceutically acceptable salt thereof wherein
R₁ is Cl, Br, CN, optionally substituted phenyl, or optionally substituted 2-, 3- or 4-pyridyl;
R₂ is optionally substituted lower alkyl, or optionally substituted aralkyl;
R₃ and R₅ are each independently optionally substituted lower alkyl, optionally substituted aryl, or optionally substituted heterocyclo;
R₄ is hydrogen or lower alkyl;
Z₁ is CO, SO₂, CO₂ or SO₂N(R₅)—; and
n is 1 or 2,
   or pharmaceutically acceptable salts thereof,
   and a BCR/ABL inhibitor or pharmaceutically acceptable salt thereof.

In another embodiment, the present invention is directed to a combination wherein the BCR/ABL inhibitor is selected from the compound of formula (I) imatinib, AMN-107, SKI 606, AZD0530, and AP23848 (ARIAD).

In another embodiment, the present invention is directed to a combination wherein the BCR/ABL inhibitor is the compound of formula (1).

In another embodiment of the invention, the invention is directed to a method of treating cancer which comprises administering, in combination, to a host in need thereof a therapeutically effective amount of:

(a) a compound of formula (II) or the compound of formula (III); and

(b) at least one compound selected from the group BCR/ABL inhibitors.

In another embodiment, the present invention is directed to a method of treating CML and/or ALL.

In another embodiment, the present invention is directed to a method of treating cancer, wherein the BCR/ABL in inhibitor is a compound of formula (I) 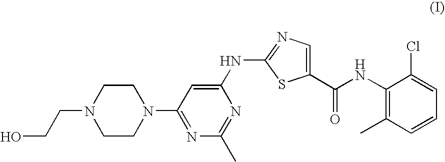
or a pharmaceutically acceptable salt or hydrate thereof.

In another embodiment, the present invention is directed to a method of treating cancer wherein the BCR/ABL inhibitor is selected from the compound of formula (I), imatinib, AMN-107, SKI 606, AZD0530, and AP23848 (ARIAD).

In another embodiment, the present invention is directed to a pharmaceutical composition comprising a therapeutically effective amount, either alone or in combination, of a compound of formula (II) or a compound of formula (III) or pharmaceutically acceptable salt thereof, and an BCR/ABL inhibitor.

In another embodiment, the present invention is directed to a pharmaceutical kit useful for the treatment of cancer, which comprises a therapeutically effective amount of:

(a) a compound of formula (II) or a compound of formula (III), or pharmaceutically acceptable salt thereof; and,

(b) at least one compound selected from the group BCR/ABL inhibitors.

In another embodiment, the present invention is directed to a pharmaceutical kit wherein the BCR/ABL inhibitor is selected from the compound of formula (I), imatinib, AMN-107, SKI 606, AZD0530, and AP23848 (ARIAD).

In another embodiment, the present invention is directed to a kit for treating CML and/or ALL.

In another embodiment of the invention, the BCR/ABL inhibitor is the compound of formula (I).

In another embodiment of the invention, the invention is directed to a combination of stem cell selective cytotoxic agents,

or pharmaceutically acceptable salts thereof,

and an BCR/ABL inhibitor or pharmaceutically acceptable salt thereof.

In another embodiment of the invention, the invention is directed to a combination of neoplastic stem cell (leukemic stem cell) selective cytotoxic agents,

or pharmaceutically acceptable salts thereof,

and an BCR/ABL inhibitor or pharmaceutically acceptable salt thereof.

In another embodiment of the invention, the invention is directed to a method of treating cancer which comprises administering, in combination, to a host in need thereof a therapeutically effective amount of:

(a) a stem cell selective cytotoxic agent; and,

(b) at least one compound selected from the group BCR/ABL inhibitors.

In another embodiment, the present invention is directed to a pharmaceutical composition comprising a therapeutically effective amount, either alone or in combination, of a stem cell selective cytotoxic agent or pharmaceutically acceptable salt thereof, and an BCR/ABL inhibitor.

In another embodiment, the present invention is directed to a pharmaceutical kit useful for the treatment of cancer, which comprises a therapeutically effective amount of:

(a) a stem cell selective cytotoxic agent, or pharmaceutically acceptable salt thereof; and,

(b) at least one compound selected from the group BCR/ABL inhibitors.

In another embodiment, the present invention is directed to a combination of the compounds of formula (II) and/or (III) with BCR/ABL inhibitors, wherein the compounds of formula (II) and/or (III) are FT inhibitors and/or RabGGTase inhibitor.

In another embodiment, the stem cell selective cytotoxic activity and the BCR/ABL activity may be present in a single compound exhibiting both activities.

In another embodiment of the invention, the invention is directed to the use of

(a) a stem cell selective cytotoxic agent; and,

(b) at least one compound selected from the group BCR/ABL inhibitors; in the manufacture of a medicament for the treatment of cancer.

In another embodiment, the invention is directed to a combination comprising

(a) a stem cell selective cytotoxic agent; and,

(b) at least one compound selected from the group BCR/ABL inhibitors;

as a combined preparation for simultaneous, separate or sequential use in therapy.

In another embodiment of the invention, the invention is directed to the use of a stem cell selective cytotoxic agent in the manufacture of a medicament for the treatment of cancer wherein the patient is also receiving treatment with at least one compound selected from the group BCR/ABL inhibitors.

In another embodiment of the invention, the invention is directed to the use of at least one compound selected from the group BCR/ABL inhibitors in the manufacture of a medicament for the treatment of cancer wherein the patient is also receiving treatment with a stem cell selective cytotoxic agent.

The invention may be embodied in other specific forms without departing from the spirit or essential attributes thereof. This invention also encompasses all combinations of preferred aspects of the invention noted herein. It is understood that any and all embodiments of the present invention may be taken in conjunction with any other embodiment to describe additional even more preferred embodiments of the present invention. Furthermore, any elements of an embodiment are meant to be combined with any and all other elements from any of the embodiments to describe additional embodiments.

### Definitions

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is hereby incorporated by reference.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

The compounds to be employed in the combination may additionally exist in a solvate, hydrate or polymorphic form. The use of such other forms are intended to be included in the present invention.

“Therapeutically effective amount” is intended to include an amount of a compound of the present invention alone or an amount of the combination of compounds claimed or an amount of a compound of the present invention in combination with other active ingredients effective to treat cancer in a host. The amount of each compound of the combination may be selected so that when the combination is administered, the effect of the combination is effective to treat cancer in a host.

As used herein, “treating” or “treatment” cover the treatment of a disease-state in a mammal, particularly in a human, and include: (a) preventing the disease-state from occurring in a mammal, in particular, when such mammal is predisposed to the disease-state but has not yet been diagnosed as having it; (b) inhibiting the disease-state, i.e., arresting it development; and/or (c) relieving the disease-state, i.e., causing regression of the disease state.

“Stem Cells” are rare quiescent cells that are capable of self renewing and maintaining tumor growth and heterogeneity. In one embodiment, “Stem cell selective cytotoxic agent” is an agent which kills the stem cells while not killing the proliferating cells.

Other features of the invention will become apparent in the course of the following descriptions of exemplary embodiments that are given for illustration of the invention and are not intended to be limiting thereof.

BCR/ABL kinase inhibitors such as the compound of formula (I) and imatinib prove to be highly effective against PH-positive/dependent CML and ALL leukemia, inducing complete cytogenetic response in the majority of patients. However, with imatinib, few patients achieve complete molecular remission. Residual disease, manifest as PCT positivity, is evident in most patients. This has been ascribed to the presence of quiescent (non-proliferating) primitive leukemic stem cells which are resistant to the cell-killing effects of BCR/ABL inhibition. There is evidence of the resistance of non-proliferating leukemic cells and primitive stem cells, respectively, to BCR/ABL inhibitors such as the compound of formula (I) and imatinib.

The major concern in the treatment of CML is resistance to the approved agent imatinib mesylate at all stages of disease, most commonly due to mutations in BCR-ABL (but other mechanisms have also been identified). Experimental agents such as dasatinib (BMS-354825), a novel, oral, multi-targeted kinase inhibitor of BCR-ABL and SRC kinases, or AMN107, which targets BCR-ABL but not SRC, were designed to address all or parts of these mechanisms and are currently under clinical testing. A second concern in CML is persistence of BCR-ABL-positive cells or ‘residual disease’ in the majority of patients on imatinib therapy, including those with complete cytogenetic responses. Bone marrow studies reveal that the residual disease resides at least in part in the primitive CD34+ progenitor compartment, suggesting that imatinib may not be effective against these cell populations (Bhatia et al, Blood 101:4701, 2003). Moreover, several imatinib-resistant ABL kinase domain mutations have been detected in CD34+/BCR-ABL+ progenitors (Chu et al, Blood 105:2093, 2005), a scenario for eventual disease relapse. A hallmark of CD34+ primitive CML progenitors is quiescence (Elrick et al, Blood 105:1862, 2005).

We hypothesized that BCR-ABL inhibitors such as imatinib may not be effective in killing CML cells in this non-proliferative state. This was tested by comparing cytotoxicity of imatinib or dasatinib in proliferating K562 cells and in cells forced into quiescence by nutrient depletion. Cytotoxicity was assessed by colony formation. Proliferating K562 cells were effectively killed by imatinib (IC50 250-500 nM) and dasatinib (IC50<1.00 nM). However, cells in the quiescent cultures were far more resistant (imatinib IC50>5000 nM; dasatinib IC50>12 nM), suggesting that these inhibitors may be less effective in eradicating quiescent CD34+ progenitors.

BMS-214662 is a FTI in Phase I clinical development. Unlike many other FTI, BMS-214662 exhibits potent cytotoxic activity against a variety of human tumor cells, and uniquely, its cytotoxicity is highly selective against non-proliferating cancer cells of epithelial origin (Lee et al, Proceedings of the AACR 42:260s, 2001).

We now demonstrate similar selectivity in K562 CML cells. BMS-214662 was 68-fold more potent in killing quiescent (IC50=0.7 uM) than proliferating K562 cells (IC50=47.5 uM). Because BCR-ABL inhibitors and BMS-214662 target distinct cell populations (proliferating vs. quiescent), there may be a positive therapeutic interaction when these agents are used in combination. In vitro studies in quiescent K562 cultures demonstrated that the combination of BMS-214662 and dasatinib, at concentrations readily achievable in the clinic, produced supra-additive cytotoxicity (% cell kill: dasatinib alone=0%, BMS-214662 alone=21%, combination=71%). In vivo studies against K562 xenografts implanted SC in SCID mice also showed that the combination of BMS-214662 and dasatinib produced a superior anti-leukemic activity than either dasatinib alone (P=0.0157) or BMS-214662 alone (P=0.0002). These results highlight the potential utility of BMS-214662 for targeting the quiescent progenitor compartment which, in combination with targeted agents such as dasatinib, address both BCR-ABL-dependent and -independent mechanisms of resistance, and may produce more durable responses and suppress the emergence of resistance.

The extent of selectivity of the two or more anti-cancer agents that comprise the method of the instant invention provide therapeutic advantages over previously disclosed methods of using a single antineoplastic agent for the treatment of cancer. In particular, use of two or more independent pharmaceutically active components that have complementary, essentially non-overlapping activities allows the person utilizing the instant method of treatment to independently and accurately vary the activity of the combination without having to synthesize a single drug having a particular pharmaceutical activity profile. In addition, such combinations should effectively target both proliferative and non-proliferative cells.

The BCR/ABL inhibitors, may be administered simultaneously with or prior to, or after the formula II compound or the compound of formula (II). In one embodiment of the present invention, the BCR/ABL inhibitor is administered prior to the formula I compound. As used herein, the term “simultaneous” or “simultaneously” means that the BCR/ABL inhibitor and the formula II compound or the compound of formula (III) are administered within 24 hours, within 12 hours, within 6 hours, or within 3 hours or less, or substantially at the same time, of each other.

In addition to the combination of the compound of formula (II) or the compound of formula (III) and the BCR/ABL inhibitors described above, the combination may be administered additionally in combination with at least one additional agent selected from the group consisting of an anti-proliferative cytotoxic agent, and an anti-proliferative cytostatic agent, and/or agents which cause cells to become “non-proliferative” or “quiescent,” referred to herein as “anti-proliferative cytostatic agents” or “quiescence agents,” may optionally be administered to a patient in need thereof. The anti-proliferative cytostatic agents may be administered simultaneously or sequentially with the combination described above or the radiation therapy or cytotoxic agent(s).

An embodiment of the present invention provides methods for the treatment and/or synergistic treatment of a variety of cancers, including, but not limited to, the following:
carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, and skin (including squamous cell carcinoma);
hematopoietic tumors of lymphoid lineage including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, histiocytic lymphoma, and Burkitts lymphoma;
hematopoietic tumors of myeloid lineage including acute and chronic myelogenous leukemias, myelodysplastic syndrome, myeloid leukemia, and promyelocytic leukemia;
tumors of the central and peripheral nervous system including astrocytoma, neuroblastoma, glioma, and schwannomas;
tumors of mesenchymal origin including fibrosarcoma, rhabdomyoscarcoma, and osteosarcoma; and
other tumors including melanoma, xeroderma pigmentosum, keratoacanthoma, seminoma, thyroid follicular cancer, and teratocarcinoma.

The invention is used to treat accelerated or metastatic cancers of the bladder, pancreatic cancer, prostate cancer, non-small cell lung cancer, colorectal cancer, and breast cancer.

The present invention provides methods for the treatment and/or synergistic treatment of a variety of non-cancerous proliferative diseases. The combination is useful to treat GIST, Breast cancer, pancreatic cancer, colon cancer, NSCLC, CML, and ALL (acute lymphoblastic leukemia, or Philadelphia chromosome positive acute lymphoblastic leukemia), sarcoma, and various pediatric cancers.

The combinations of the present invention are useful for the treatment of cancers such as chronic myelogenous leukemia (CML), gastrointestinal stromal tumor (GIST), small cell lung cancer (SCLC), non-small cell lung cancer (NSCLC), ovarian cancer, melanoma, mastocytosis, germ cell tumors, acute myelogenous leukemia (AML), pediatric sarcomas, breast cancer, colorectal cancer, pancreatic cancer, prostate cancer and others known to be associated with protein tyrosine kinases such as, for example, SRC, BCR-ABL and c-KIT. The compounds of the present invention are also useful in the treatment of cancers that are sensitive to and resistant to chemotherapeutic agents that target BCR-ABL and c-KIT, such as, for example, Gleevec® (imatinib, STI-571).

As used herein, the phrase “radiation therapy” includes, but is not limited to, x-rays or gamma rays which are delivered from either an externally applied source such as a beam or by implantation of small radioactive sources. Radiation therapy may also be considered an anti-proliferative cytotoxic agent.

As used herein, the phrase “anti-neoplastic agent” is synonymous with “chemotherapeutic agent” and refers to compounds that prevent cancer cells from multiplying (i.e. anti-proliferative agents). In general, the agent(s) of this invention fall into two classes, anti-proliferative cytotoxic and anti-proliferative cytostatic. Cytotoxic agents prevent cancer cells from multiplying by: (1) interfering with the cell's ability to replicate DNA and (2) inducing cell death and/or apoptosis in the cancer cells. Anti-proliferative cytostatic or quiescent agents act via modulating, interfering or inhibiting the processes of cellular signal transduction which regulate cell proliferation. The majority of chemotherapeutic agents are cytotoxic and target proliferating cells.

Agents which may be used in combination with the present combination are described in WO2005/013983, which is hereby incorporated by reference in its entirety.

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR), e.g., 1996 edition (Medical Economics Company, Montvale, N.J. 07645-1742, USA); the disclosure of which is incorporated herein by reference thereto.

An embodiment of the present invention also encompasses a pharmaceutical composition useful in the treatment of cancer, comprising the administration of a therapeutically effective amount of the combinations of this invention, with or without pharmaceutically acceptable carriers or diluents. The pharmaceutical compositions of this invention comprise the compound of formula II, the compound of formula (III), and/or the stem cell selective cytotoxic agent, and a BCR/ABL inhibitor. The pharmaceutical composition of this invention additionally comprise an optional anti-proliferative cytotoxic agent or agents, an optional quiescence agent, and a pharmaceutically acceptable carrier. The compositions of the present invention may further comprise one or more pharmaceutically acceptable additional ingredient(s) such as alum, stabilizers, antimicrobial agents, buffers, coloring agents, flavoring agents, adjuvants, and the like. The compounds of the combination of the present invention and compositions of the present invention may be administered orally or parenterally including the intravenous, intramuscular, intraperitoneal, subcutaneous, rectal and topical routes of administration.

For oral use, the compounds of the combination and compositions of this invention may be administered, for example, in the form of tablets or capsules, powders, dispersible granules, or cachets, or as aqueous solutions or suspensions. In the case of tablets for oral use, carriers which are commonly used include lactose, corn starch, magnesium carbonate, talc, and sugar, and lubricating agents such as magnesium stearate are commonly added. For oral administration in capsule form, useful carriers include lactose, corn starch, magnesium carbonate, talc, and sugar. When aqueous suspensions are used for oral administration, emulsifying and/or suspending agents are commonly added. In addition, sweetening and/or flavoring agents may be added to the oral compositions. For intramuscular, intraperitoneal, subcutaneous and intravenous use, sterile solutions of the active ingredient(s) are usually employed, and the pH of the solutions should be suitably adjusted and buffered. For intravenous use, the total concentration of the solute(s) should be controlled in order to render the preparation isotonic. In another embodiment of the present invention, the compounds of the combination or pharmaceutically acceptable salts thereof are formulated with a sulfobutylether-7-β-cyclodextrin or a 2-hydroxypropyl-β-cyclodextrin for intravenous administration.

For preparing suppositories according to the invention, a low melting wax such as a mixture of fatty acid glycerides or cocoa butter is first melted, and the active ingredient is dispersed homogeneously in the wax, for example by stirring. The molten homogeneous mixture is then poured into conveniently sized molds and allowed to cool and thereby solidify.

Liquid preparations include solutions, suspensions and emulsions. Such preparations are exemplified by water or water/propylene glycol solutions for parenteral injection. Liquid preparations may also include solutions for intranasal administration.

Aerosol preparations suitable for inhalation may include solutions and solids in powder form, which may be in combination with a pharmaceutically acceptable carrier, such as an inert compressed gas.

Also included are solid preparations which are intended for conversion, shortly before use, to liquid preparations for either oral or parenteral administration. Such liquid forms include solutions, suspensions and emulsions.

The compounds of the combination described herein may also be delivered transdermally. The transdermal compositions can take the form of creams, lotions, aerosols and/or emulsions and can be included in a transdermal patch of the matrix or reservoir type as are conventional in the art for this purpose.

The combinations may also be used in conjunction with other well known therapies that are selected for their particular usefulness against the condition that is being treated.

If formulated as a fixed dose, the active ingredients of the combination compositions of this invention are employed within the dosage ranges known to one skilled in the art. Alternatively, the compounds of the combination may be administered separately in the appropriate dosage ranges.

An embodiment of the present invention is directed to a combination of the compound of formula (II) or the compound of formula (III), (the compound of Formula III being an FTI inhibitor, but the activity of the compound may not be dependent on the specific mechanism of action) which is a quiescent cell selective cytotoxic agent and which may be useful as a stem cell selective cytotoxic agent, and an BCR/ABL inhibitor. The BCR/ABL inhibitors such as the compound of formula (I) and imatinib are known to treat proliferating cancer cells and therefore are effective in the treatment of cancers such as CML and ALL. However, the BCR/ABL inhibitors such as the compound of formula (I) and imatinib are known to not affect quiescent and stem cells. Therefore, the combination of the quiescent cell selective cytotoxic agent or the stem cell selective cytotoxic agent with the BCR/ABL inhibitor is useful in eliminating or eradicating residual disease which are drug resistant leukemic stem cells.

Examples of BCR/ABL inhibitors, include, but are not limited to, the compound of formula (I), imatinib (Gleevec®, STI-571, Novartis), AMN-107 (Novartis), SKI 606 (Schering Plough), AZD0530 (Astra Zeneca), and AP23848 (ARIAD). Other BCR/ABL inhibitors may be identified by methods known to those of skill in the art.

An embodiment of the present invention is further directed to the a combination of the compound of formula (II), or pharmaceutically acceptable salts thereof, and the compound of formula (I), or pharmaceutically acceptable salt, and/or hydrate, thereof.

An embodiment of the present invention is further directed to the a method of treating CML and/or ALL comprising administering the combination of the compound of formula (II) and the compound of formula (I). The invention is further embodied by the combination of a quiescent cell selective cytotoxic agent or stem cell selective cytotoxic agents in combination with a BCR/ABL inhibitor (wherein the BCR/ABL inhibitor may be a Src inhibitor and/or a BCR/ABL inhibitor). Quiescent cell selective cytotoxic agents are represented by the compounds of formula (II) and (III). Additional stem cell selective cytotoxic agents may be identified by as described below.

Stem Cells Isolation:

Pluripotent Ph+ stem cells are primitive, quiescent and remain cytokine non-responsive for several days in culture. In growth factor supplemented serum free cultures, using CFSE to track cell division, CD34 to track differentiation and annexin V to track apoptosis, the non-proliferating, CD34+ CML stem cells can be isolated by flurorescence-activated cell sorting technique (Erlick et al. 2004, BLOOD prepublished online Nov. 4, 2004).

The stem cells would then be treated with the agent being studied to determine if the agent killed the stem cells.

Study Design and Methodology:

K562 cells were maintained in RPMI-1640 and 10% FCS
Proliferating (P) cells are defined as cells in exponential growth phase obtained on Day 2 following culture initiation on Day 0 at a concentration of 3×104 cells/mL
Quiescent (Q) cells are defined as cells in stationary growth phase obtained on Day 8 following culture initiation at a concentration of 3×104 cells/mL with no medium change

Other methods are detailed in individual figure legends in the Results section

Results:

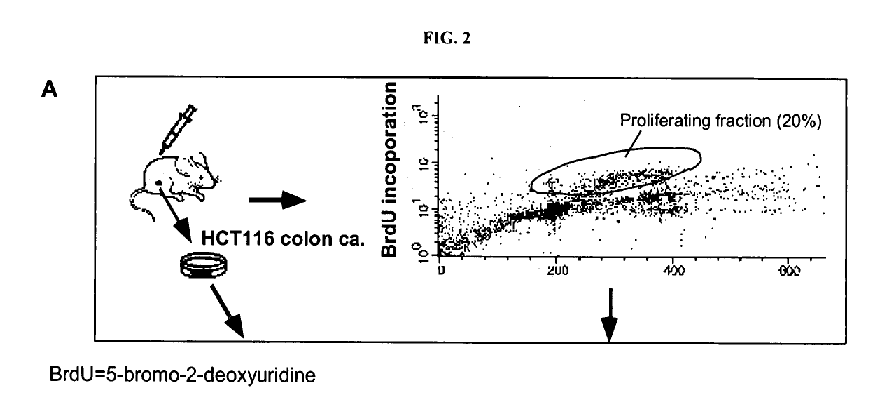

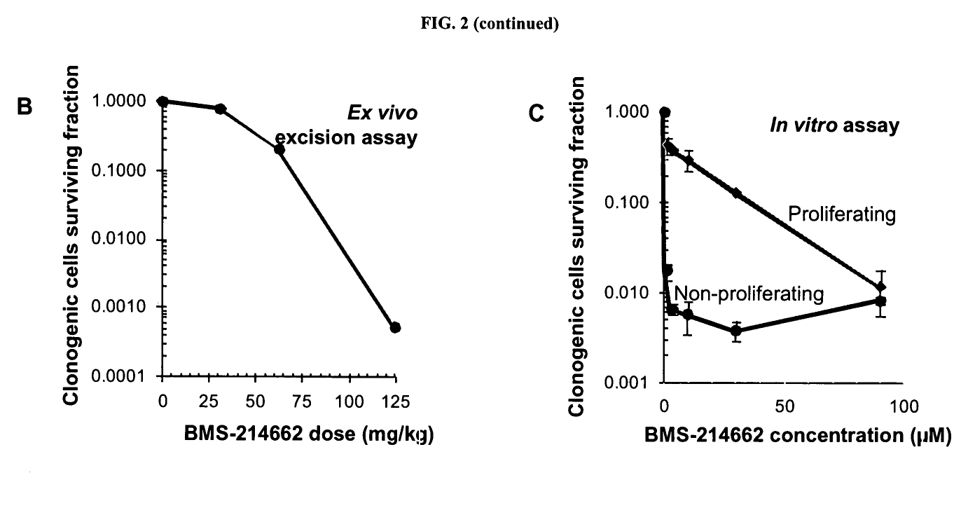

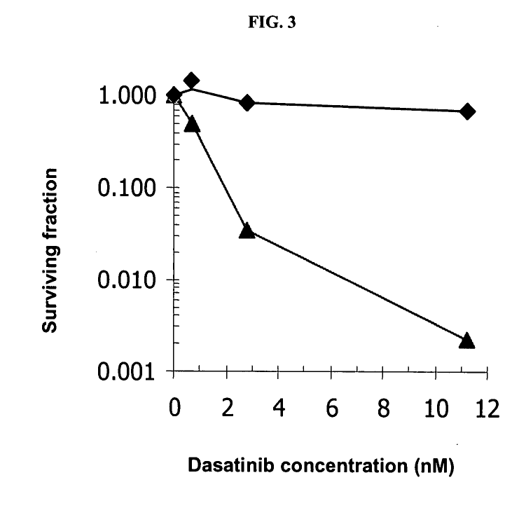

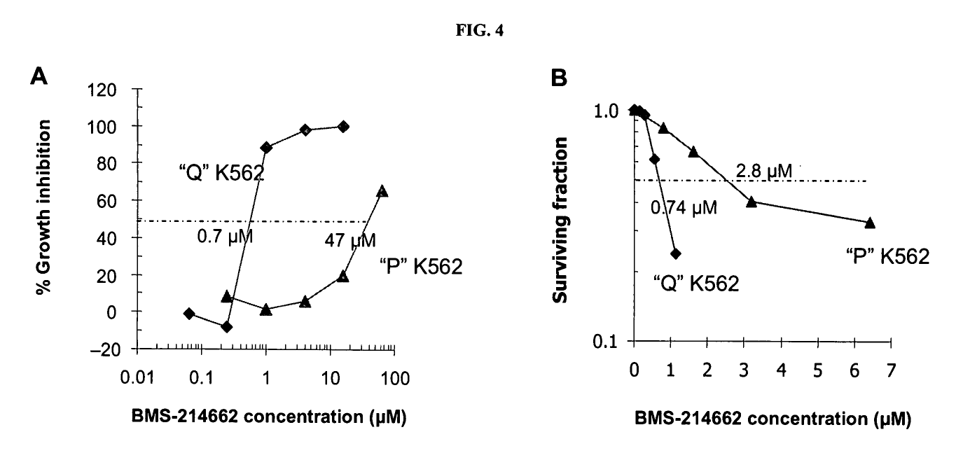

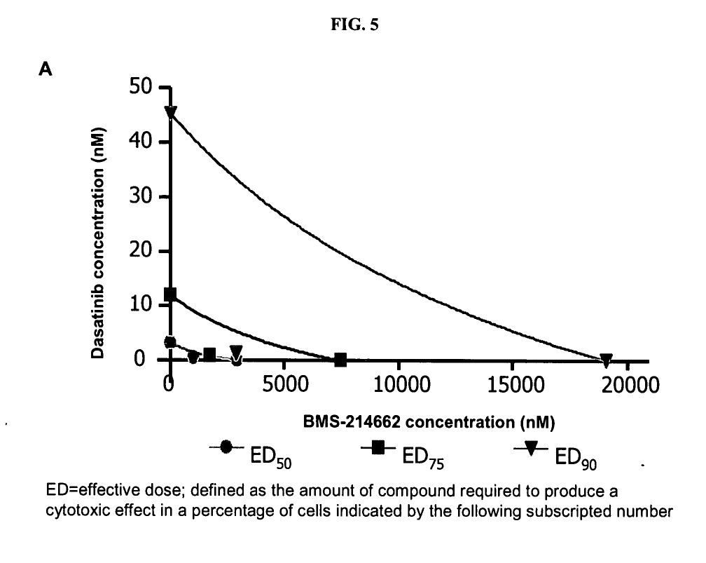

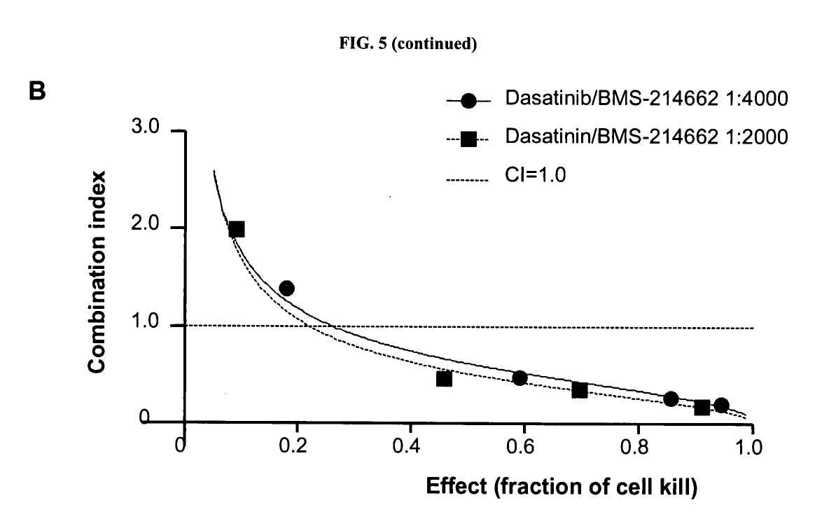

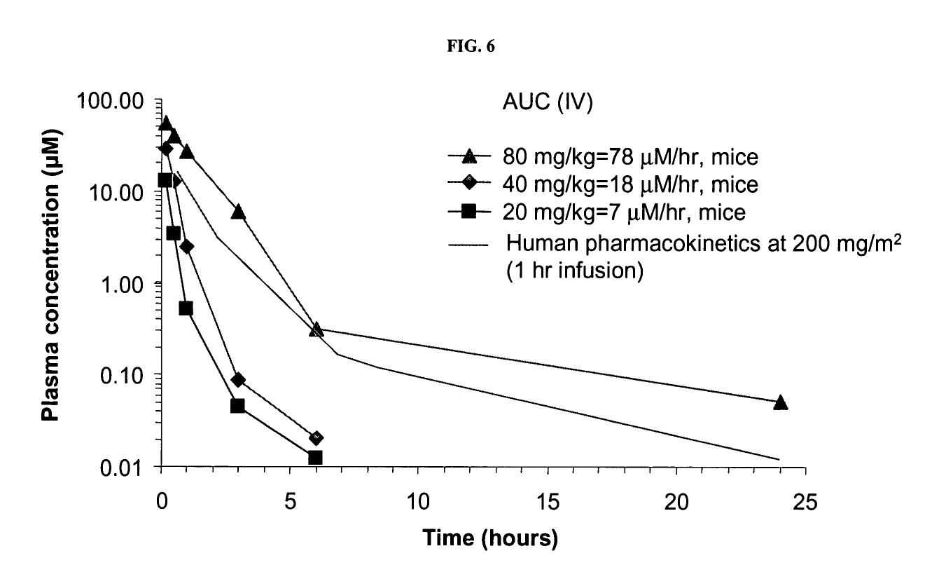

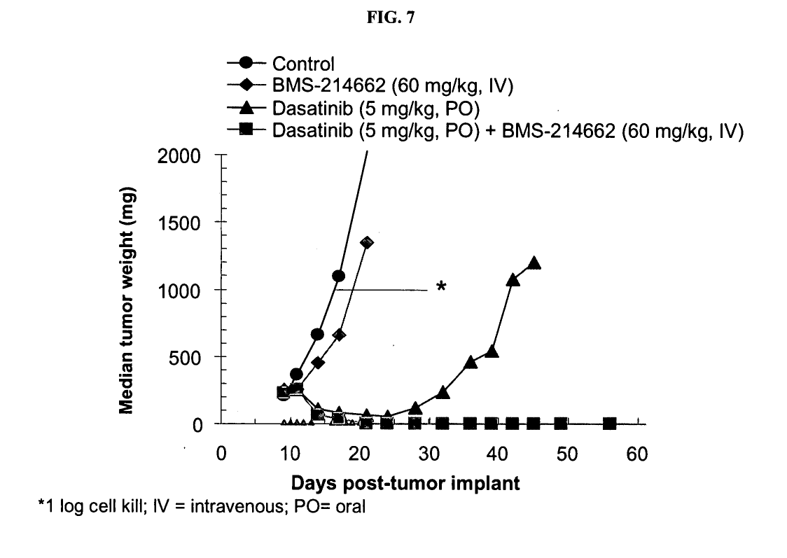

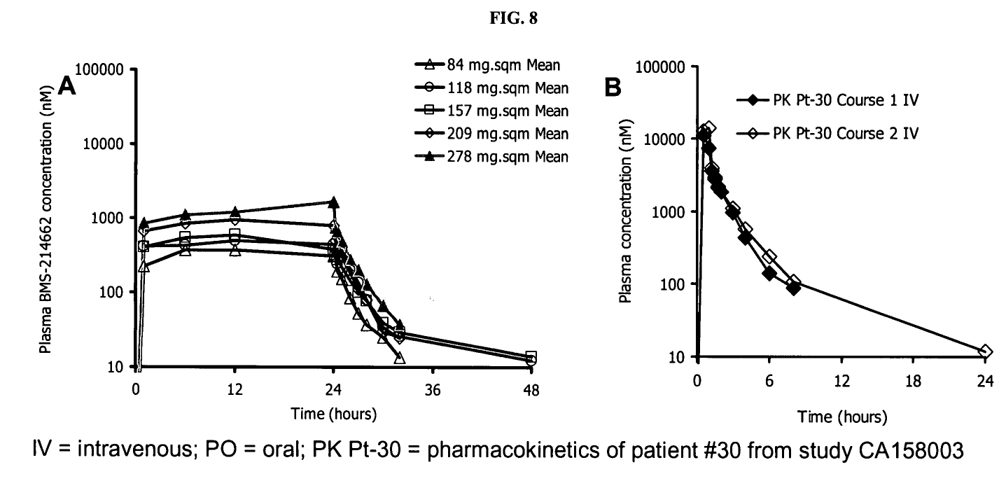

FIG. 2. BMS-214662 affords massive killing of clonogenic tumor cells in vivo, and is specific for non-proliferating cells. (A) Analysis of tumors xenografts by FACS analysis demonstrated that only 20% of tumor cells were proliferative. The vast majority of the tumor cells were in the non-proliferative (G0) growth stage. Non-proliferative cells were identified by prolonged BrdU labeling (24 h) of tumor cells within a solid tumor by continuous infusion of mice bearing the HCT-116 human colon carcinoma subcutaneously in vivo. (B) BMS-214662 killed >90% of clonogenic cells, the vast majority of which would be non-proliferating. (C) BMS-214662 has greater cell killing potency in quiescent than in proliferating cells.
Dasatinib is a more potent agent than BMS-214662 in the management of imatinib-sensitive and -resistant CML, but does not eradicate non-proliferating stem cells
BMS-214662 preferentially acts against non-proliferative versus proliferative leukemic stem cells
The combination of dasatinib and BMS-214662 is highly synergistic, both in vitro and in vivo
The plasma levels required for BMS-214662 to enhance the anti-leukemic activity of dasatinib are achievable clinically
These results highlight the potential therapeutic utility of BMS-214662 for targeting quiescent leukemic stem cells, in combination with dasatinib, which targets both BCR-ABL-dependent and -independent mechanisms of imatinib resistance, in the management of CML
Dasatinib monotherapy Phase II trials in imatinib-resistant/-intolerant CML and Philadelphia-chromosome positive acute lymphoblastic leukemia (Ph+ ALL)—the ‘START’ program—have now closed; initial data will be presented at this congress, and extended follow-up continues.

### REFERENCES

1. Schindler T et al. Science 2000; 289:1938-42
2. Gorre M E et al. Science 2001; 293:876-80
3. Shah N P et al. Science 2004; 305:399-401
4. Bhatia R et al. Blood 2003; 101:4701-7
5. Li S et al. ASH annual meeting 2005; Poster presentation 1990
6. Chu S et al. Blood 2005; 105:2093-8
7. Elrick L J et al. Blood 2005; 105:1862-6
8. O'Hare T et al. Cancer Res 2005; 65:4500-5
9. Shah N P et al. Science 2004; 305:399-401
10. Talpaz M et al. J Clin Oncol 2005; 23(16S):564s (Abstract 6519)
11. Sawyers C L et al. J Clin Oncol 2005; 23(16S):565s (Abstract 6520)
12. Peng C et al. ASH annual meeting 2005; Poster presentation 2861
13. Copland M et al. ASH annual meeting 2005; Oral presentation 695
14. Lee F Y F et al. Proceedings of the AACR 2001; 42:260s
15. Copland M et al. ASH annual meeting 2005; Oral presentation 693

What is claimed is:

1. A method of treating cancer which comprises administering, in combination, to a host in need thereof a therapeutically effective amount of:
(a) a stem cell selective cytotoxic agent or pharmaceutically acceptable salt thereof, and
(b) at least one of a BCR/ABL inhibitor or pharmaceutically acceptable salt thereof.

2. The method of claim 1, wherein the stem cell selective cytotoxic agent is a compound of formula (III) 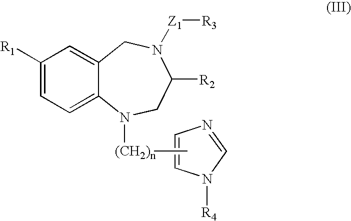
or a pharmaceutically acceptable salt thereof wherein
R₁ is Cl, Br, CN, optionally substituted phenyl, or optionally substituted 2-, 3- or 4-pyridyl;
R₂ is optionally substituted lower alkyl, or optionally substituted aralkyl;
R₃ and R₅ are each independently optionally substituted lower alkyl, optionally substituted aryl, or optionally substituted heterocyclo;
R₄ is hydrogen or lower alkyl;
Z₁ is CO, SO₂, CO₂ or SO₂N(R₅)—; and
n is 1 or 2.

3. The method of treating cancer of claim 2, wherein the compound of formula (III), is selected from a compound of formula (II) 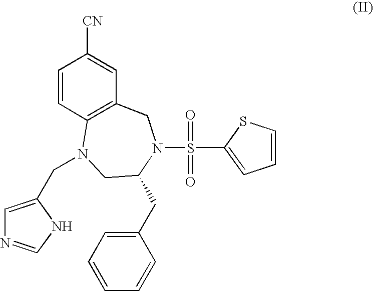
or pharmaceutically acceptable salts thereof.

4. The method of treating cancer of claim 2, wherein the BCR/ABL inhibitor is selected from the compound of formula (I) 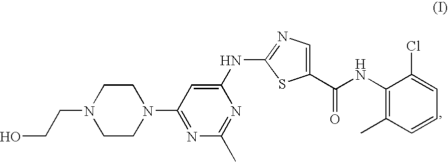
imatinib, AMN-107, SKI 606, AZD0530, and AP23464, or a pharmaceutically acceptable salt or hydrate thereof.

5. The method of treating cancer of claim 1, wherein the cancer is selected from chronic myelogenous leukemia (CML) and Philadelphia chromosome positive acute lymphoblastic leukemia (ALL).

6. A combination which comprises a therapeutically effective amount of:
(a) a stem cell selective cytotoxic agent or pharmaceutically acceptable salt thereof, and
(b) at least one of a BCR/ABL inhibitor or pharmaceutically acceptable salt thereof.

7. The combination of claim 6, wherein the stem cell selective cytotoxic agent is a compound of formula (III) 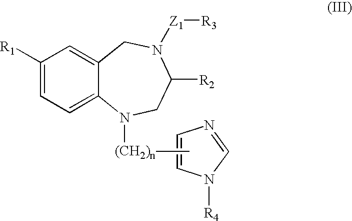
or a pharmaceutically acceptable salt thereof wherein
R₁ is Cl, Br, CN, optionally substituted phenyl, or optionally substituted 2-, 3- or 4-pyridyl;
R₂ is optionally substituted lower alkyl, or optionally substituted aralkyl;
R₃ and R₅ are each independently optionally substituted lower alkyl, optionally substituted aryl, or optionally substituted heterocyclo;
R₄ is hydrogen or lower alkyl;
Z₁ is CO, SO₂, CO₂ or SO₂N(R₅)—; and
n is 1 or 2.

8. The combination of claim 7, wherein the compound of formula (III), is selected from a compound of formula (II) 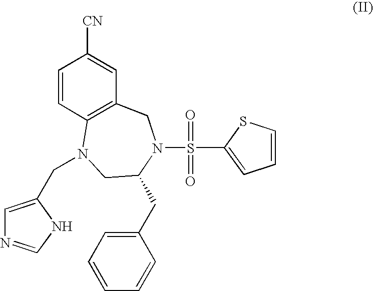
or pharmaceutically acceptable salts thereof.

9. The combination of claim 2, wherein the BCR/ABL inhibitor is selected from the compound of formula (I) 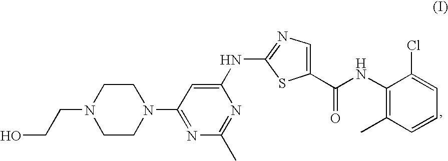
imatinib, AMN-107, SKI 606, AZD0530, and AP23464, or a pharmaceutically acceptable salt or hydrate thereof.

10. A method of treating cancer which comprises administering, in combination, to a host in need thereof a therapeutically effective amount of:
(a) a compound of formula (II) 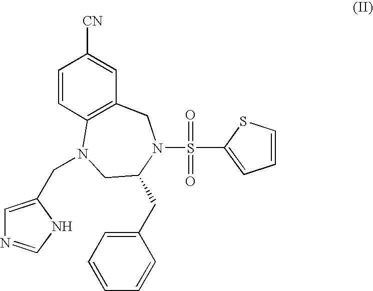
or pharmaceutically acceptable salts thereof, and
(b) and a compound of formula (I) 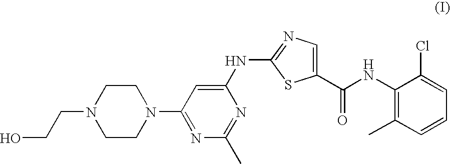
or pharmaceutically acceptable salts or hydrate thereof.

11. A pharmaceutical composition, comprising a pharmaceutically acceptable vehicle or diluent and at least one of each of the compounds of the combination of claim 6.
